SEQUENCE LISTING (26 sequences)

SEQ ID NO: 1 - DNA, 1170 bp, Brassica napus
  source 1..1170: mol_type = genomic DNA
  CDS 1..1170
atggcggcgcagggaaactccacggacacacttttctctccttacaagatgggaagattcgatctctctcacagagtggttctggcgccgatgacacgttgcagggcgttgaacggcgttccgaacgcggcgttggcggagtattacgctcaacgcaccactcccggcggttttctcatctccgaaggcaccatgatctctcccggatccgcagggtttccacatgtaccaggaatctattcagaggaacaagtagaggcatggaaacccgttgtggaagcagttcactccaaaggaggtctcatcttctgtcaactatggcatgttggacgagcttctcatccgttgtaccaacctaatggtggatcgccaatatcgtcaacggacaaagccatctcagaaagatggcgagttctgttgcctgatggatcacacgcgaaataccctaaacctcgacctctacacgcttccgagatacctcgagtggtggaagattattgcaactcggctattaatgcaatccgagctggttttgatgggattgagatccatggagcgcatggttacctcattgatcagtttatgaaaaatgggatcaatgaccgtaccgaccaatatggaggctccattcaaaaccgttgcagattcttacttcaggtagtgaaaggtgtggtttcagccataggagctagtaaagttggcgtcaggatatctccagcaatagaccatctcgatgcaactgattctgacccactctccctcgggctagccgtggttgacttgctcaacaagttacaggacgatactggctccaagctcgcttaccttcacgtaacacagcctcggtaccacgcttatgggcaaacagaatctggaaggcaaggcagtgatgaggaagaagctaagttaatgaagagcttgcgtacggcttacaatgggacatttatgtctagtggaggtttcaacaaggaacttggtatgcaagccattcagcaaggggatgctgatttggtctcctttggcaggctgtttattgcaaaccctgacttggtttcgcggtttaaggttggtgcgaagttgaataaatataatcggaagacgttttacactcaagaccccgttgttggctacacagattattccttcttggctccagcttctcgcctctga

SEQ ID NO: 2 - protein, 389 aa, Brassica napus
MAAQGNSTDTLFSPYKMGRFDLSHRVVLAPMTRCRALNGVPNAALAEYYAQRTTPGGFLISEGTMISPGSAGFPHVPGIYSEEQVEAWKPVVEAVHSKGGLIFCQLWHVGRASHPLYQPNGGSPISSTDKAISERWRVLLPDGSHAKYPKPRPLHASEIPRVVEDYCNSAINAIRAGFDGIEIHGAHGYLIDQFMKNGINDRTDQYGGSIQNRCRFLLQVVKGVVSAIGASKVGVRISPAIDHLDATDSDPLSLGLAVVDLLNKLQDDTGSKLAYLHVTQPRYHAYGQTESGRQGSDEEEAKLMKSLRTAYNGTFMSSGGFNKELGMQAIQQGDADLVSFGRLFIANPDLVSRFKVGAKLNKYNRKTFYTQDPVVGYTDYSFLAPASRL

SEQ ID NO: 3 - DNA, 1170 bp, Brassica napus
  source 1..1170: mol_type = genomic DNA
atggcggcgcagggaaactccacggacacacttttctctccttacaagatgggaagattcgatctctctcacagagtggttctggcgccgatgacacgttgcagggctttgaacggcgttccgaacgcggctttggcggagtattacgctcaacgcaccactcccggcggttttctcatctccgaaggcaccatgatctctcccggatccgcagggttcccacatgtaccaggaatctattccgaggaacaggtagaagcatggaagcacgtcgtggaagcagttcactccaaaggaggtctcatcttctgtcaattatggcatgttggacgagcttctcatccgttgtaccaacccaacggtggatcgccaatatcgtcaacggacaaacccatctcagaaagatggcgagttctgttgcccgatggatcacacgcgaaataccctaaacctcggcctctgcacgcttctgagatacctcgagtggtggaagattattgcaactcggctgttaacgcaatccgagctggttttgatgggattgagatccatggagcgcacggttacctcattgatcagtttatgaaaaatgggatcaatgaccgtaccgaccaatatggtggctccattgaaaaccgttgcagattcttgcttcaggtagtgaaaggtgtggtttcagcgataggagcgagtaaagttggcgtgaggatatctccagcaatagaccatctcgatgcaactgattccgacccactctccctcgggctagccgtggttgacttgctcaacaagttacaggacgatactggctccaagctcgcttaccttcatgtaacacagcctcggtaccacgcttacgggcaaacagaatctggaaggcaaggcagtgatgaggaagaagctaagttaatgaagagcttgcgtacggcttacaatggaacatttatgtcaagtggaggtttcaacaaggaacttggtatgcaagccattcggcaaggggatgctgatttggtttcctttgggaggctgtttattgcaaaccctgatttggtttcgcggtttaaggttggtgcgaagttgaataaatataatcggaagacgttttacactcaagaccccgttgttggctacacagattattccttcttggctccagtttctcgcctctga

SEQ ID NO: 4 - protein, 389 aa, Brassica napus
MAAQGNSTDTLFSPYKMGRFDLSHRVVLAPMTRCRALNGVPNAALAEYYAQRTTPGGFLISEGTMISPGSAGFPHVPGIYSEEQVEAWKHVVEAVHSKGGLIFCQLWHVGRASHPLYQPNGGSPISSTDKPISERWRVLLPDGSHAKYPKPRPLHASEIPRVVEDYCNSAVNAIRAGFDGIEIHGAHGYLIDQFMKNGINDRTDQYGGSIENRCRFLLQVVKGVVSAIGASKVGVRISPAIDHLDATDSDPLSLGLAVVDLLNKLQDDTGSKLAYLHVTQPRYHAYGQTESGRQGSDEEEAKLMKSLRTAYNGTFMSSGGFNKELGMQAIRQGDADLVSFGRLFIANPDLVSRFKVGAKLNKYNRKTFYTQDPVVGYTDYSFLAPVSRL

SEQ ID NO: 5 - DNA, 20 bp, synthetic construct
  source 1..20: mol_type = other DNA
tggaaggcaaggcagtgatg

SEQ ID NO: 6 - DNA, 24 bp, synthetic construct
  source 1..24: mol_type = other DNA
attgtggaaggcaaggcagtgatg

SEQ ID NO: 7 - DNA, 24 bp, synthetic construct
  source 1..24: mol_type = other DNA
aaaccatcactgccttgccttcca

SEQ ID NO: 8 - DNA, 23 bp, synthetic construct
  source 1..23: mol_type = other DNA
gttcacattagctatgtcatttt

SEQ ID NO: 9 - DNA, 23 bp, synthetic construct
  source 1..23: mol_type = other DNA
cgaacaagcaggccacacaggat

SEQ ID NO: 10 - DNA, 23 bp, synthetic construct
  source 1..23: mol_type = other DNA
gttcatattagctatgtcactga

SEQ ID NO: 11 - DNA, 20 bp, synthetic construct
  source 1..20: mol_type = other DNA
gaaacacgaacaagcaggat

SEQ ID NO: 12 - DNA, 25 bp, synthetic construct
  source 1..25: mol_type = other DNA
tgtcccaggattagaatgattaggc

SEQ ID NO: 13 - DNA, 25 bp, synthetic construct
  source 1..25: mol_type = other DNA
ccccagaaattgaacgccgaagaac

SEQ ID NO: 14 - DNA, 18 bp, synthetic construct
  source 1..18: mol_type = other DNA
gatggattgcacgcaggt

SEQ ID NO: 15 - DNA, 19 bp, synthetic construct
  source 1..19: mol_type = other DNA
tcgtcaagaaggcgataga

SEQ ID NO: 16 - DNA, 17 bp, synthetic construct
  source 1..17: mol_type = other DNA
acgagcgccatccgatc

SEQ ID NO: 17 - DNA, 21 bp, synthetic construct
  source 1..21: mol_type = other DNA
gttcaccctgaggatgtcgct

SEQ ID NO: 18 - DNA, 23 bp, Brassica napus
  source 1..23: mol_type = genomic DNA
tggaaggcaaggcagtgatgagg

SEQ ID NO: 19 - DNA, 24 bp, Brassica napus
  source 1..24: mol_type = genomic DNA; note = base A in position 18 is insterted
tggaaggcaaggcagtgaatgagg

SEQ ID NO: 20 - DNA, 24 bp, Brassica napus
  source 1..24: mol_type = genomic DNA; note = base A in position 18 is inserted
tggaaggcaaggcagtgaatgagg

SEQ ID NO: 21 - DNA, 24 bp, Brassica napus
  source 1..24: mol_type = genomic DNA; note = base T in position 18 is inserted
tggaaggcaaggcagtgtatgagg

SEQ ID NO: 22 - DNA, 24 bp, Brassica napus
  source 1..24: mol_type = genomic DNA; note = base T in position 18 is inserted
tggaaggcaaggcagtgtatgagg

SEQ ID NO: 23 - DNA, 24 bp, Brassica napus
  source 1..24: mol_type = genomic DNA; note = base A in position 18 is inserted
tggaaggcaaggcagtgaatgagg

SEQ ID NO: 24 - DNA, 24 bp, Brassica napus
  source 1..24: mol_type = genomic DNA; note = base T in position 18 is inserted
tggaaggcaaggcagtgtatgagg

SEQ ID NO: 25 - DNA, 24 bp, Brassica napus
  source 1..24: mol_type = genomic DNA; note = base A in position 18 is inserted
tggaaggcaaggcagtgaatgagg

SEQ ID NO: 26 - DNA, 20 bp, Brassica napus
  source 1..20: mol_type = genomic DNA; note = 3 bases are deleted in positions 18-20
tggaaggcaaggcagtgagg

(19) United States
(12) Patent Application Publication
(10) Pub. No.: US 2026/0109996 A1
(43) Pub. Date: Apr. 23, 2026

(54) METHOD FOR CREATING MALE STERILE AND MAINTAINER DUAL-PURPOSE LINE BY KNOCKING OUT BnOPR3 GENE IN BRASSICA NAPUS

(71) Applicant: Oil Crops Research Institute, CAAS, Wuhan (CN)

(72) Inventors: Hongtao CHENG, Wuhan (CN); Qiong Hu, Wuhan (CN); Mengyu HAO, Wuhan (CN); Desheng MEI, Wuhan (CN); Hui WANG, Wuhan (CN); Wenxiang WANG, Wuhan (CN); Yunfei WEN, Wuhan (CN); Li FU, Wuhan (CN); Jia LIU, Wuhan (CN); Chao LI, Wuhan (CN)

(21) Appl. No.: 18/921,256

(22) Filed: Oct. 21, 2024

(51) Int. Cl. C12N 15/82; C12N 9/22

(52) U.S. Cl. CPC C12N 15/8289; C12N 9/22; C12N 15/8201; C12N 2310/20

(57) ABSTRACT

A method for creating a male sterile and maintainer dual-purpose line by knocking out a BnOPR3 gene in Brassica napus, belongs to the technical field of plant genetic engineering. Use of two Brassica napus homologous genes of the BnOPR3 gene in controlling male reproductive development of Brassica napus is provided, where a BnOPR3.A3 gene has a nucleotide sequence set forth in SEQ ID NO: 1, and a protein encoded by same has a sequence set forth in SEQ ID NO: 2; and a BnOPR3.C3 gene has a nucleotide sequence set forth in SEQ ID NO: 3, and a protein encoded by same has a sequence set forth in SEQ ID NO: 4. A method for creating a male sterile and a maintainer dual-purpose line using a BnOPR3 gene in Brassica napus is further provided.

### REFERENCE TO SEQUENCE LISTING

A computer readable XML file entitled “GWP20240806212-SEQUENCE LISTING”, that was created on Oct. 16, 2024, with a file size of about 28,494 bytes, contains the sequence listing for this application, has been filed with this application, and is hereby incorporated by reference in its entirety.

### TECHNICAL FIELD

The present disclosure belongs to the technical field of crop molecular breeding, and specifically relates to a method for creating a male sterile and maintainer dual-purpose line by knocking out a BnOPR3 gene in Brassica napus.

### BACKGROUND

Male sterile line is important for utilizing hybrid vigor and hybrid seed production in crops, mainly includes cytoplasmic male sterility (CMS) and genic male sterility (GMS). CMS is controlled together by mitochondrial genes and nuclear genes. Although being applied to the breeding and hybrid seed production of crops such as Brassica napus, CMS still has problems such as cytoplasm type of sterile line is relatively simple and susceptibility to diseases. GMS is controlled by nuclear genes alone and can overcome the defects of CMS, but is difficult to mass-produce homozygous sterile lines through conventional breeding methods. Both CMS and GMS involve three-line matching and require seed production fields and breeding fields. The emergence of photoperiod- and thermo-sensitive genic male sterile (PGMS and TGMS) lines has enabled the three-line breeding to be simplified into two-line breeding, which exhibits certain production and utilization value. A two-line method combines the maintainer line and the sterile line into one. This method is not restricted by a restorer-maintainer relationship, and is easy to obtain a strong advantage combination by random hybridization. In this way, a male sterile line of Brassica napus with stable and reliable fertility is created, and there are no 50% fertile plants during the breeding of the sterile line, and there is no need to manually remove the fertile plants, either. These methods may bring a historic breakthrough to the production of hybrid Brassica napus.

It has been reported in many literatures that clustered regularly interspaced short palindromic repeats/CRISPR-associated protein 9 (CRISPR/Cas9) system-mediated gene editing has been effectively applied in the creation of plant sterile lines, e.g. TMS5 was knocked out in japonica rice by CRISPR/Cas9-mediated gene editing to develop a transgene-free TGMS line (Zhou et al., 2016); a PGMS mutant was obtained by knocking out the CARBON-STARVED ANTHERS gene in japonica rice (Li et al., 2016; Gu et al., 2019); Singh et al. performed CRISPR/Cas9 editing on the homologous genes from three homologous chromosomes A, B and D of wheat Ms45, and mutants with simultaneous mutations in all the three homeologs showed male sterility (Singh et al., 2018); targeted mutation of the homologous gene of OsNP1 in alfalfa also led to the generation of GMS lines (Ye et al., 2021). CRISPR/Cas9-mediated gene editing was performed on multiple homologous genes required for pollen development and male reproduction in maize, including ZmDFR1, ZmDFR2, ZmACOS5-1 and ZmACOS5-2, to obtain a new maize male sterile line (Liu et al., 2022). Therefore, it is a convenient and effective means by creating plant male sterile lines through the gene editing.

Compared with the model plant Arabidopsis thaliana and rice, there are relatively few GMS genes cloned and identified as well as male sterile materials created in Brassica napus. CRISPR/Cas9-mediated gene editing is increasingly being used in plant gene function research, crop genetic improvement, and breeding due to low cost, easy operation, and high mutation induction rate, showing an extremely broad application prospect. The excavation and identification of male sterile candidate genes in Brassica napus using CRISPR/Cas9 to create male sterile materials can quickly enrich Brassica napus GMS gene and sterile material resources, thereby promoting the application of hybrid vigor in Brassica napus. Ultimately, it is possible to effectively break through the bottleneck of lacking stable Brassica napus sterile lines and breakthrough major varieties that rapeseed industry has long faced in worldwide.

So far, there has been no report on the BnOPR3 gene being related to male fertility in Brassica napus, nor has there been any report on use of the gene and the CRISPR/Cas9 system to create male sterile lines in Brassica napus. There is no report on use of the gene to create mutants to achieve “one line for two purposes” in Brassica napus and create novel Brassica napus germplasm.

### SUMMARY

In view of the shortcomings in the prior art, an objective of the present disclosure is to provide a Brassica napus BnOPR3 gene and use thereof in creating a Brassica napus male sterile line. The use can overcome the problem that 50% of fertile plants in the breeding of the GMS male sterile line in the existing hybrid breeding of Brassica napus need to be manually pulled out. The present disclosure provides a method for creating a male sterile and maintainer dual-purpose line, thereby realizing the creation of novel Brassica napus germplasm materials with “one line for two purposes”.

To achieve the above objective, the present disclosure provides use of a BnOPR3 gene in controlling male reproductive development of Brassica napus. Generally, it can be expected that OPR3 homologous genes from different plants or different Cruciferae materials have the same or similar functions, and thus these genes can also be used to control plant fertility. Moreover, even if the functions of these genes cannot be predicted, a person skilled in the art can determine whether they have the function of controlling plant male fertility based on the method provided by the present disclosure and the prior art.

The present disclosure further provides use of two Brassica napus homologous genes of the BnOPR3 gene in controlling male reproductive development of Brassica napus, where a BnOPR3.A3 gene has a coding sequence set forth in SEQ ID NO: 1, and a protein coded by same has an amino acid sequence set forth in SEQ ID NO: 2; and a BnOPR3.C3 gene has a coding sequence set forth in SEQ ID NO: 3, and a protein coded by same has an amino acid sequence set forth in SEQ ID NO: 4.

In another aspect, the present disclosure further provides a method for creating a male sterile line of Brassica napus, including inhibiting expression and/or an activity of the BnOPR3 gene in the Brassica napus, and selecting a male sterile plant of the Brassica napus.

In some embodiments, the method is any one selected from the group consisting of gene editing, RNA interference, and T-DNA insertion.

In some embodiments, the gene editing is CRISPR/Cas9.

In some embodiments, the CRISPR/Cas9 includes: designing a CRISPR/Cas9 vector target site at the exon 5 on the BnOPR3.A3 gene or the BnOPR3.C3 gene, where the vector target site has a DNA sequence set forth in SEQ ID NO: 5.

In another aspect, the present disclosure further provides a method for preparing a male sterile line of a BnOPR3 gene, including: obtaining the male sterile line of the BnOPR3 gene by the method described above, hybridizing and backcrossing the male sterile line to a target material, such that the target material acquires a male sterile trait of the BnOPR3 gene and achieves gene mutation.

The present disclosure further provides use of the male sterile line of the BnOPR3 gene prepared by the method in hybrid breeding and seed producing of Brassica napus, where during the hybrid breeding and the seed producing, the male sterile line of the BnOPR3 gene is used as a female parent to allow the hybridization with other male parents; alternatively, hybridizing and backcrossing the male sterile line of the BnOPR3 gene with other target materials, such that the other target materials acquire the male sterile trait of the BnOPR3 gene and achieves the gene mutation.

The advantages and beneficial effects of the present disclosure are as follows:

The BnOPR3 gene and the protein coded by same in regulating male reproductive development of Brassica napus have not been reported before. In the present disclosure, both the BnOPR3.A3 and BnOPR3.C3 genes in Brassica napus are mutated by CRISPR/Cas9. It is found that only simultaneous mutation of the above two homologs of the BnOPR3 gene can cause male sterility in Brassica napus, while a single mutation has no effect on the development of anthers and pollen in Brassica napus. CRISPR/Cas9-mediated gene editing and a male sterile mutant of the BnOPR3 gene obtained after editing can be used to create male sterile lines of Brassica napus, which are used in hybrid breeding and seed production of Brassica napus. The mutant material is treated with methyl jasmonate (MeJA) at an appropriate concentration in the late stage of Brassica napus buds. The male fertility of the BnOPR3 gene mutant can be restored, and a resulting next generation can maintain the sterility trait, that is, conversion between the sterile line and the maintainer line is achieved to overcome the defects of traditional artificial emasculation.

### BRIEF DESCRIPTION OF THE DRAWINGS

FIG. 1 is a diagram showing the structure of the BnOPR3 gene and the position of sgRNA;

FIG. 2 shows a schematic diagram of a gene editing vector;

FIG. 3 shows sequences of homozygous mutants of the BnOPR3 gene;

FIGS. 4A-4B show sterility phenotypes of a double-copy homozygous mutant and a single-copy homozygous mutant of the BnOPR3 gene; where FIG. 4A is a double-copy homozygous mutant of the BnOPR3 gene showing complete sterility; and FIG. 4B is a single-copy homozygous mutant of the BnOPR3 gene showing normal fertility;

FIGS. 5A-5D show section images of the double-copy homozygous mutant of the BnOPR3 gene and control anthers; where FIGS. 5A and 5C are cross-sectional views of wild-type recepient anthers at two developmental stages; and FIGS. 5B and 5D are cross-sectional views of anthers of double-copy homozygous mutant of the BnOPR3 gene at two developmental stages; the arrows indicate locations of the anther cell crack;

FIGS. 6A-6C show fertility phenotypes of a double-copy homozygous mutant of the BnOPR3 gene after spraying with MeJA; where FIG. 6A is a fertility phenotype of a double-copy homozygous mutant of the BnOPR3 gene after spraying with MeJA and water; FIG. 6B is a magnified view of the flower before treatment; and FIG. 6C is a magnified view of the flower after spraying with MeJA and water;

FIG. 7 shows fruiting of the double-copy homozygous mutant of the BnOPR3 gene after spraying with MeJA;

FIGS. 8A-8B show phenotypes of F₁ progeny obtained by crossing the double-copy homozygous mutant of the BnOPR3 gene with a normal material; where FIG. 8A shows phenotypes of the two parents and the F₁ hybrid at the flowering stage; and FIG. 8B shows phenotypes of the two parents and the F₁ hybrid at the late flowering stage;

FIGS. 9A-9F show plant height and yield traits of the hybrid F₁ generation; where FIG. 9A is a diagram showing plant height of the parents and hybrids; FIG. 9B is a diagram showing number of branches of the parents and hybrids; FIG. 9C is a diagram showing number of siliques per plant of the parents and hybrids; FIG. 9D is a diagram showing length of siliques of the parents and hybrids; FIG. 9E is a diagram showing thousand-seed weight of the parents and hybrids; and FIG. 9F is a diagram showing yield per plant of the parents and hybrids; and

FIGS. 10A-10C show a sterility phenotype of the self-pollinated F₁ progeny of a double-copy homozygous mutant of the BnOPR3 gene after spraying with MeJA; where FIG. 10A is a plant phenotype of the self-pollinated F₁ progeny of the double-copy homozygous mutant of the BnOPR3 gene after spraying with MeJA; FIG. 10B and FIG. 10C are magnified views of flowers of the plant.

### DETAILED DESCRIPTION OF THE EMBODIMENTS

### Description of the Sequence Listing

SEQ ID NO: 1 is the CDS of the BnOPR3.A3 gene.

SEQ ID NO: 2 is the amino acid sequence coded by the BnOPR3.A3 gene.

SEQ ID NO: 3 is the CDS of the BnOPR3.03 gene.

SEQ ID NO: 4 is the amino acid sequence coded by the BnOPR3.C3 gene.

SEQ ID NO: 5 is a sgRNA sequence designed in the present disclosure.

SEQ ID NO: 6 is one of two oligo DNA single-stranded sequences synthesized in the present disclosure.

SEQ ID NO: 7 is the other oligo DNA single-stranded sequences synthesized in the present disclosure.

SEQ ID NO: 8 is a forward primer sequence for amplifying an A3 copy of the BnOPR3 gene.

SEQ ID NO: 9 is a reverse primer sequence for amplifying the A3 copy of the BnOPR3 gene.

SEQ ID NO: 10 is a forward primer sequence for amplifying a C3 copy of the BnOPR3 gene.

SEQ ID NO: 11 is a reverse primer sequence for amplifying the C3 copy of the BnOPR3 gene.

SEQ ID NO: 12 is a forward primer sequence for amplification and identification the E. coli positive clones.

SEQ ID NO: 13 is a reverse primer sequence for amplification and identification the E. coli positive clones.

SEQ ID NO: 14 is a forward primer sequence for the detection of whether the T₀ transformed plants carried exogenous selection markers.

SEQ ID NO: 15 is a reverse primer sequence for the detection of whether the T₀ transformed plants carried exogenous selection markers.

SEQ ID NO: 16 is a forward primer sequence for Cas9 gene identification.

SEQ ID NO: 17 is a reverse primer sequence for Cas9 gene identification.

SEQ ID NO: 18 is a sequence of sgRNA and PAM motif in the conserved region of the genome of Brassica napus.

SEQ ID NO: 19 is a sequence of the homologous copy of BnOPR3 gene of L105-32 plant on A03 chromosome.

SEQ ID NO: 20 is a sequence of the homologous copy of BnOPR3 gene of L105-32 plant on (03 chromosome.

SEQ ID NO: 21 is a sequence of the homologous copy of BnOPR3 gene of L2-8 plant on A03 chromosome.

SEQ ID NO: 22 is a sequence of the homologous copy of BnOPR3 gene of L2-8 plant on (03 chromosome.

SEQ ID NO: 23 is a sequence of the homologous copy of BnOPR3 gene of L106-8 plant on A03 chromosome.

SEQ ID NO: 24 is a sequence of the homologous copy of BnOPR3 gene of L106-8 plant on A03 chromosome.

SEQ ID NO: 25 is a sequence of the homologous copy of BnOPR3 gene of L106-8 plant on (03 chromosome.

SEQ ID NO: 26 is a sequence of the homologous copy of BnOPR3 gene of L106-8 plant on (03 chromosome.

The present disclosure is described in detail below with reference to examples, which are not intended to limit the scope of the present disclosure. Modifications or substitutions made to methods, steps or conditions of the present disclosure without departing from the concept of the present disclosure fall within the scope of the present disclosure.

Unless otherwise particularly specified, the instruments, agents, materials and the like involved in the following examples are existing common instruments, agents, materials and the like in the prior art, and are commercially available. Unless otherwise particularly specified, the experimental methods, test methods and the like involved in the following examples are existing common experimental methods, test methods and the like in the art.

### Example 1: Design of sgRNA and Construction of Genome Editing Vector for BnOPR3 Gene of Brassica napus Based on CRISPR/Cas9

1. Determination of sgRNA Sequence

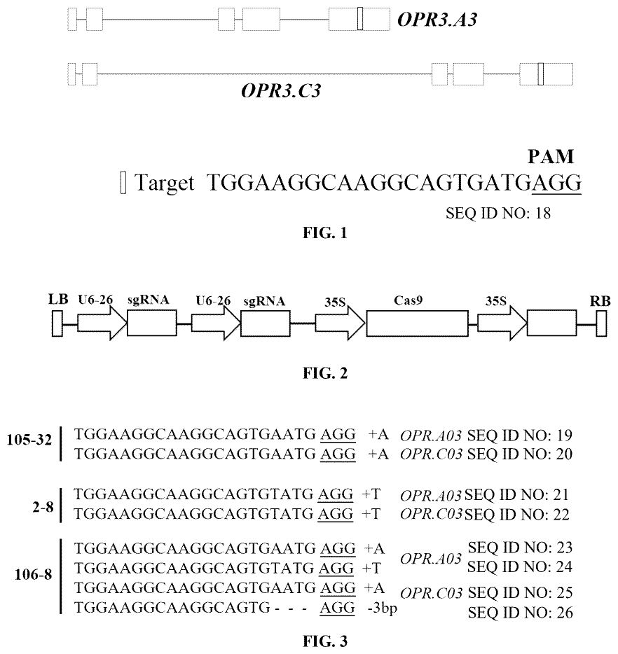

Two copies of the BnOPR3 gene of Brassica napus were aligned, a PAM motif (NGG) was searched for in a conserved region, and a sequence 20 bp upstream of a 5′-end of the PAM position was the sgRNA sequence. A sgRNA was designed with a sequence set forth in SEQ ID NO: 5 and a target site set forth in FIG. 1.

### 2. Synthesis of Oligo DNA Single Strand

The sequences of the two oligo DNA single strands were set forth in SEQ ID NO: 6 and SEQ ID NO: 7, which were synthesized by Sangon Biotech (Shanghai) Co., Ltd.

3. Construction of the Genome Editing Vector pKSE401-BnOPR3

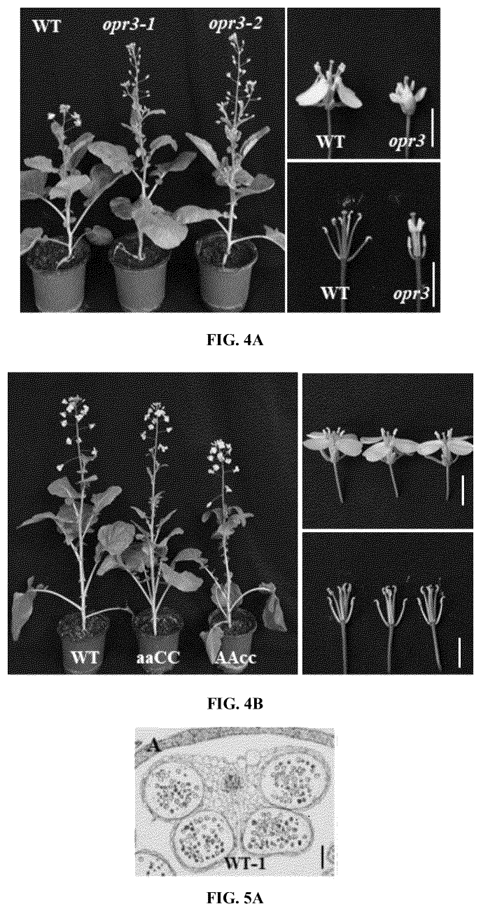

The two oligo DNA single strands were being treated at 95° C. for 5 min, then cooled naturally. The primer pairs were ligated to a CRISPR/Cas9 vector (FIG. 2), the components were mixed well on ice according to the following reaction system, reacted at 37° C. for 5 h and at 50° C. for 5 min, and then placed at 80° C. for 10 min to terminate the reaction.

The reaction system included: 2 μL primer pairs+2 μL of pKSE401+1.5 μL of 10×T4 DNA Ligase Buffer (NEB)+1.5 μL of 10×BSA+1 μL of BsaI (NEB)+1 μL of T4 DNA Ligase (NEB)+6 μL of ddH₂O.

4. Escherichia coli Transformation

5 μL of a ligation product was added to 50 μL of E. coli DH5α competent cells and mixed well; a resulting mixture was treated in ice bath for 30 min avoiding shaking during this period; after heat shock at 42° C. for 45 s, the mixture was immediately put on ice for 2 min; 400 μL of LB liquid medium was added, and posttransformation culture was conducted at 37° C., 200 rpm for not less than 30 min; 100 μL of a resulting bacterial solution was spread on LB medium containing 50 mg/L kanamycin, and cultured at 37° C. overnight.

### 5. Positive Clone Identification and Plasmid Extraction

A single clone was selected and inoculated into LB liquid medium containing 50 mg/L kanamycin; cultured at 37° C., 200 rpm for 5 h, 1 μL of a resulting bacterial solution was taken for PCR identification using primers U6-26 PF: 5′-TGTCCCAGGATTAGAATGATTAGGC-3′ (SEQ ID NO: 12) and U6-26t-R: 5′-CCCCAGAAATTGAACGCCGAAGAAC-3′ (SEQ ID NO: 13); and the confirmed positive clones by Sanger sequencing were subjected to plasmids extraction by conventional alkaline lysis method.

### Example 2: Transformation of Agrobacterium GV3101 Using the Target Vector

1 μL of target vector plasmid was added to 100 μL of GV3101 Agrobacterium competent cells; after mixing well, a resulting mixture was treated in ice bath for 5 min, frozen in liquid nitrogen for 5 min, and treated in 37° C. water bath for 5 min; 700 μL of LB liquid medium was added, shaken at 28° C., 200 rpm for 3 h; an appropriate amount of resulting bacterial solution was collected and spread on LB solid medium containing 50 mg/L kanamycin, 50 mg/L gentamicin, and 50 mg/L rifampicin; after culturing in the dark at 28° C. for 48 h, a single clone was selected and inoculated in LB liquid medium containing 50 mg/L kanamycin, 50 mg/L gentamicin, and 50 mg/L rifampicin, and cultured overnight at 28° C., 200 rpm; PCR was performed with primers U6-26 PF: 5′-TGTCCCAGGATTAGAATGATTAGGC-3′ and U6-26t-R: 5′-CCCCAGAAATTGAACGCCGAAGAAC-3′ to identify the positive clone. The Agrobacterium clone that was confirmed to be transformed successfully was used for genetic transformation of Brassica napus.

### Example 3: Agrobacterium-Mediated Genetic Transformation of Brassica napus

### 1. Explant Preparation

Surface disinfection of Westar rapeseeds collected in this laboratory: the seeds were added into a clean 50 mL centrifuge tube and treated with 75% Ethanol for 1 min; the Ethanol was discarded, 10 mL of 0.15% mercuric chloride was added to immerse the seeds for 15 min, while the seeds were shaken every few minutes to make the liquid fully contact with the seeds; the mercuric chloride was discarded and the seeds were washed 4 times with sterilized single distilled water; the Westar seeds of Brassica napus were transferred to M0 medium, 20 seeds per bottle, and about 120 seeds were required for each transformation; the seeds were cultivated in the dark at 24° C. for 5 d to 6 d.

### 2. Agrobacterium Infection and Co-Culture

10 μL of the Agrobacterium culture was taken from a −80° C. ultra-low temperature refrigerator, thawed, and added into 1 mL of LB liquid medium containing 50 mg/L kanamycin, 50 mg/L gentamicin, and 50 mg/L rifampicin, incubated overnight for activation, and the activated bacterial solution was inoculated for streak cultivation on the LB solid medium containing 50 mg/L kanamycin, 50 mg/L gentamicin, and 50 mg/L rifampicin at 28° C. for 24 h to 48 h; Brassica napus seedlings cultured in the dark for 6 d were taken out, placed in a sterilized large dish and their hypocotyls were cut into sections with a sterilized scalpel, where each section was about 0.8 cm long, and a small amount of Dilution Medium (DM) was poured into the large dish to keep moist during the cutting; the Agrobacterium cells were scraped with an inoculation loop into the DM containing acetosyringon (AS) at a final concentration of 100 μM, and the Agrobacterium cells were activated at 28° C. and 200 rpm for 30 min; the cut hypocotyls were transferred to the activated Agrobacterium bacterial solution and infected for 30 min while shaking every five minutes or so during this period; explants were transferred to an empty culture dish with sterilized filter paper, spread, and the bacterial solution was dried with a small filter paper; the explants were transferred to the M1 medium and cultured in the dark at 24° C. for 48 h.

### 3. Screening and Differentiation

The explants that were co-cultured for 48 h were transferred to M2 medium containing 25 mg/L kanamycin and cultured at 24° C. for about 3 weeks with a light duration of 16 h/d; at this time, the explants were stained darker and began to swell into callus tissue. The explants were transferred to M3 medium containing 25 mg/L kanamycin and cultured at 24° C. with a light duration of 16 h/day, and the explants were subcultured every two weeks to ensure sufficient nutrition until regenerated seedlings were observed.

### 4. Rooting of Regenerated Seedlings

The regenerated seedlings were cut off from the callus tissue with forceps and a scalpel, transferred into M4 medium commonly used for plant tissue culture, and then cultured at 24° C. with a light duration of 16 h/day until rooting.

### 5. Transplanting

The regenerated seedlings with desirable rooting were transplanted into nutrient soil and covered with a lid to maintain humidity. After 2 d, the lid was opened to allow normal growth.

The medium formulas used were as follows:
Murashige and Skoog (MS) Mixture: NH₄NO₃ 1650 mg/L, H₃BO₃ 6.2 mg/L, CaCl₂ 332.2 mg/L, CoCl₂·6H₂O 0.025 mg/L, CuSO₄:5H₂O 0.025 mg/L, Na₂EDTA·2H₂O 37.26 mg/L, FeSO₄·7H₂O 27.8 mg/L, MgSO₄ 180.7 mg/L, MnSO₄·H₂O 16.9 mg/L, Na₂MoO₄·2H₂O 0.25 mg/L, KI 0.83 mg/L, KNO₃ 1900 mg/L, KH₂PO₄ 170 mg/L, ZnSO₄·7H₂O 8.6 mg/L, Glycine 2 mg/L, myo-Inositol 100 mg/L, Nicotinic Acid 0.5 mg/L, Pyridoxine·HCl 0.5 mg/L, Thiamine·HCl 0.1 mg/L
M0: MS Mixture 2.2 g/L+agar powder 7 g/L, pH=6.0
DM: MS Mixture 4.4 g/L+sucrose 30 g/L, added with 100 μM AS before use, pH=6.0
M1: MS Mixture 4.4 g/L+sucrose 30 g/L+mannitol 18 g/L+2,4-D 1 mg/L+kinetin (KT) 0.3 mg/L+agar powder 7 g/L,
added with 1 mL of 100 mM AS before use, pH=6.0
M2: MS Mixture 4.4 g/L+sucrose 30 g/L+mannitol 18 g/L+2,4-D 1 mg/L+KT 0.3 mg/L+agar powder 7 g/L, added with kanamycin to 25 mg/L, Timentin (TMT) to 300 mg/L, 1.5/1000 volume of silver thiosulfate (STS), pH=6.0
M3: MS Mixture 4.4 g/L+glucose 10 g/L+xylose 0.25 g/L+2-(4-Morpholino) ethanesulfonic acid (MES) 0.6 g/L+agar powder 7 g/L, added with kanamycin to 25 mg/L, TMT to 300 mg/L, Zeatin to 2 mg/L, pH=6.0
M4: Gamborg B5 Medium: ((NH₄)₂SO₄ 134 mg/L, H₃BO₃ 3 mg/L, CaCl₂ 113.24 mg/L, CoCl₂·6H₂O 0.025 mg/L, CuSO₄·5H₂O 0.025 mg/L, Na₂EDTA·2H₂O 37.26 mg/L, FeSO₄·7H₂O 27.8 mg/L, MgSO₄ 122.09 mg/L, MnSO₄·H₂O 10 mg/L, Na₂MoO₄·2H₂O 0.25 mg/L, KI 0.75 mg/L, KNO₃ 2500 mg/L, NaH₂PO₄ 150 mg/L, ZnSO₄·7H₂O 2 mg/L, myo-Inositol 100 mg/L, Nicotinic Acid 1 mg/L, Pyridoxine HCl 1 mg/L, Thiamine·HCl 10 mg/L) 3.21 g/L+sucrose 30 g/L+agar powder 7.5 g/L, pH=6.0.

### Example 4: Mutation Detection in Transgenic Plants

### 1. Screening and Detecting of Transformed Plants

A genomic DNA of the transgenic T₀ plants with kanamycin resistance was extracted by conventional CTAB method, and the transformed plants were detected by PCR amplification using primers: NPTII-F: 5′-GATGGATTGCACGCAGGT-3′ (SEQ ID NO: 14) and NPTII-R: 5′-TCGTCAAGAAGGCGATAGA-3′ (SEQ ID NO: 15), to detect whether the T₀ transformed plants carried exogenous selection markers.

### 2. Mutation Detection of BnOPR3-sgRNA Transformed Strain

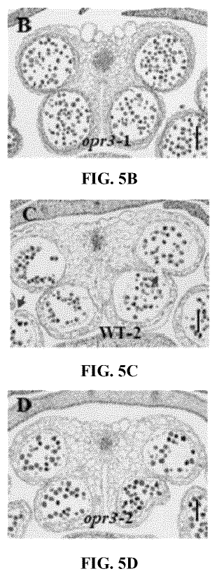

Since two copies of the BnOPR3 gene were on chromosomes A3 and C3, respectively, two pairs of primers were designed (sequences were shown in SEQ ID NOs: 8-9 and SEQ ID NOs. 10-11). The gene fragments near the target sites of BnaA3.OPR3 and BnaC3.OPR3 were amplified from the genomic DNA of the identified positive BnOPR3-sgRNA transformed plants by PCR, and PCR products were subjected to Sanger sequencing to identify whether the target sequences of the two copies A3 and C3 of the BnOPR3 genes in the transformed plants were edited. The sequencing results showed that in L2, L105, and L106 transformed plants, the BnOPR3 gene produced heterozygous mutations at the target sites of the two homologous copies of chromosomes A3 and C3. The T₁ generation materials of the above mutant families were planted to continue testing the gene editing type. The sequencing results showed that in L2-8, L105-32, and L106-8 plants, the BnOPR3 gene was homozygous for mutations at the target sites of the two homologous copies of chromosomes A3 and C3 (FIG. 3).

### Example 5: Screening of T₂ Generation Transgene-Free Strain & Plant Phenotype Observation and Trait Statistics

The BnOPR3 gene edited T₁ generation homozygous mutant strain was self-pollinated and harvested, and then sown and planted to obtain a T₂ generation. Kanamycin selection marker identification and Cas9 gene identification were conducted on the T₂ generation homozygous mutant strain using kanamycin selection marker identification primers (NPTII-F: 5′-GATGGATTGCACGCAGGT-3′, NPTII-R: 5′-TCGTCAAGAAGGCGATAGA-3′) and Cas9 gene identification primers (Cas9-F: 5′-ACGAGCGCCATCCGATC-3′ (SEQ ID NO: 16), Cas9-R: 5′-GTTCACCCTGAGGATGTCGCT-3′ (SEQ ID NO: 17)), thus screening transgene-free homozygous mutant strains.

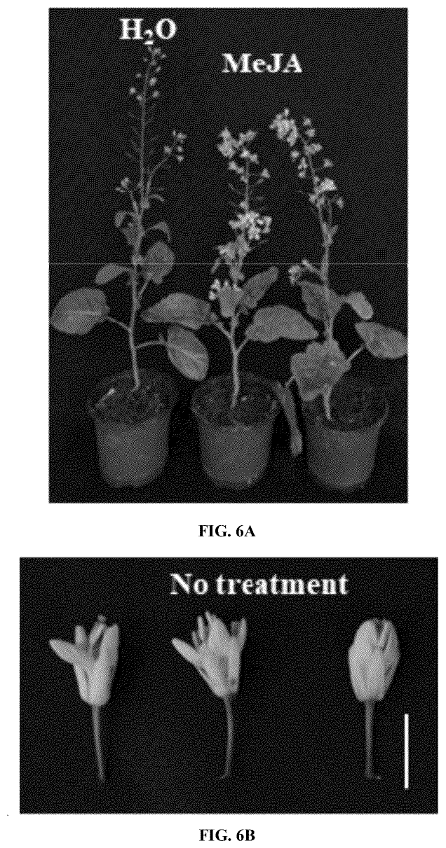

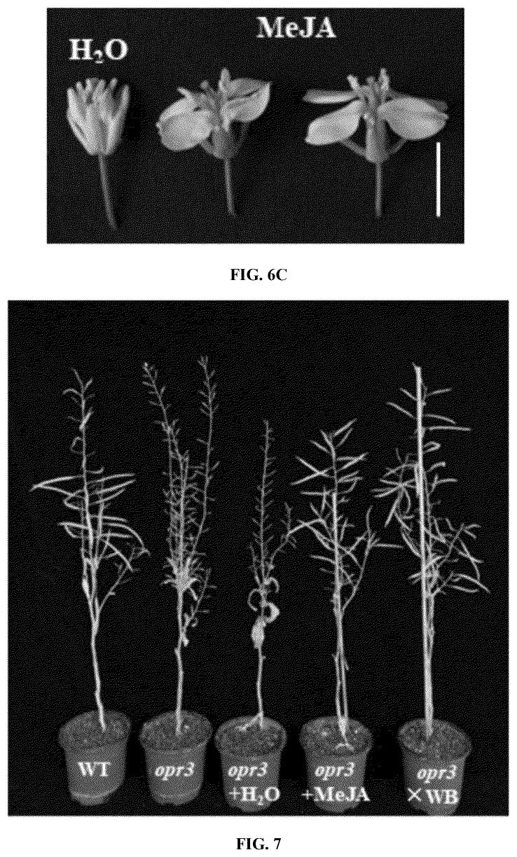

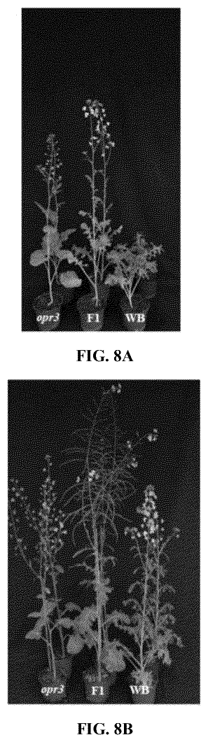

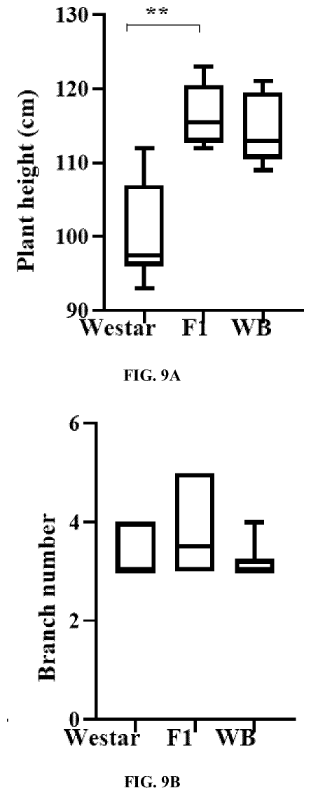

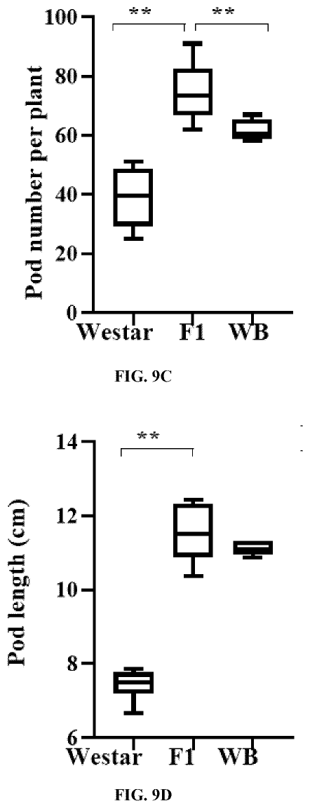

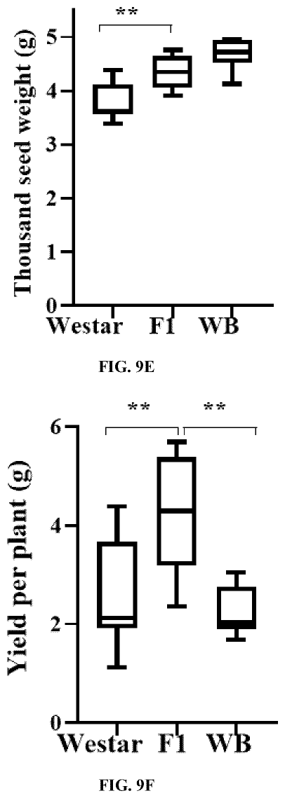

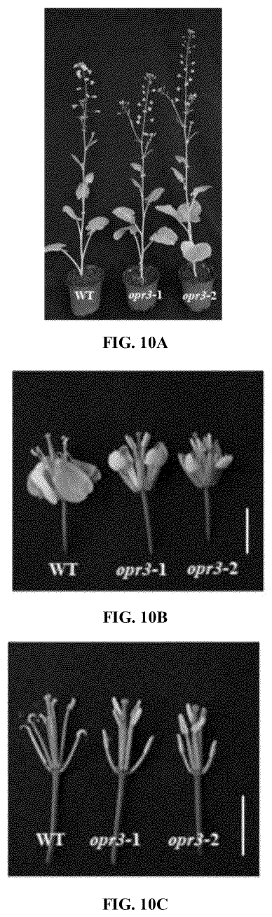

It was found in the study that the homozygous mutant with both copies of the BnOPR3 gene knocked out had no difference in phenotype before flowering compared with the wild-type material, but the BnOPR3 gene-knockout mutant showed male sterility after flowering (FIGS. 4A-4B). The mutants with any single copy knockout of the BnOPR3 gene did not show any difference in fertility compared with the wild-type (FIGS. 4A-4B). Another section analysis was conducted on the homozygous mutant with both copies of the BnOPR3 gene knocked out and the wild-type material. There was no difference in the pollen grain cell morphology between the homozygous mutant with the BnOPR3 gene knockout and the wild-type material (see FIGS. 5A-5D). The sterile materials of the BnOPR3 gene-knockout homozygous mutant were sprayed with 500 μM MeJA and water. It was found that the fertility of the materials sprayed with MeJA returned to normal and mature pollen grains released could be seen on the anthers, while the materials sprayed with water remained sterile (FIGS. 6A-6C). This indicated that spraying a certain concentration of MeJA could restore the sterile characteristics of the BnOPR3 gene-knockout homozygous mutant. The study found that the progeny of the mutant material sprayed with MeJA could bear fruit normally (see FIG. 7). When the sterile materials of the BnOPR3 gene-knockout homozygous mutant were crossed with a normal material, their progeny showed normal fruiting (see FIGS. 8A-8B); the number of siliques per plant and the yield per plant showed obvious hybrid vigor (see FIGS. 9A-9F). The progeny of the mutant material sprayed with MeJA bore fruit normally, and their obtained seed progeny also showed sterility characteristics (see FIGS. 10A-10C), indicating that the sterility of the mutant could be stably inherited. The above results suggested that the functional loss mutant strain obtained by using the CRISPR/Cas9 system to knock out the BnOPR3 gene in Brassica napus in the present disclosure. This mutant strain exhibited male sterility and its fertility could be restored by simply spraying a certain concentration of MeJA, thus being applied in Brassica napus hybrid production and hybrid vigor utilization.

### MAIN REFERENCES

1. Gu W, Zhang D, Qi Y, Yuan Z. Generating Photoperiod-Sensitive Genic Male Sterile Rice Lines with CRISPR/Cas9. Methods Mol Biol. 2019, 1917:97-107.
2. Li Q, Zhang D, Chen M, Liang W, Wei J, Qi Y, Yuan Z. Development of japonica photo-sensitive genic male sterile rice lines by editing Carbon Starved Anther using CRISPR/Cas9. J. Genet. Genom. 2016, 43:415-419.
3. Liu X, Zhang S, Jiang Y, Yan T, Fang C, Hou Q, Wu S, Xie K, An X, Wan X. Use of CRISPR/Cas9-Based Gene Editing to Simultaneously Mutate Multiple Homologous Genes Required for Pollen Development and Male Fertility in Maize. Cells. 2022, 11:439.
4. Singh M, Kumar M, Albertsen M, Young K, Cigan M. Concurrent modifications in the three homeologs of Ms45 gene with CRISPR-Cas9 lead to rapid generation of male sterile bread wheat (Triticum aestivum L.) Plant Mol. Biol. 2018, 97:371-383.
5. Xing H L, Dong L, Wang Z P, Zhang H Y, Han C Y, Liu B, Wang X C, Chen Q J. A CRISPR/Cas9 toolkit for multiplex genome editing in plants. BMC Plant Biol. 2014, 14:327.
6. Ye Q, Meng X, Chen H, Wu J, Zheng L, Shen C, Guo D, Zhao Y, Liu J, Xue Q, Dong J, Wang T. Construction of genic male sterility system by CRISPR/Cas9 editing from model legume to alfalfa. Plant Biotechnology Journal. 2022, 20:613-615.

What is claimed is:

1. A method for controlling male reproductive development of Brassica napus with a BnOPR3.A3 gene that is a homologue of BnOPR3 gene, wherein the BnOPR3.A3 gene has a coding sequence (CDS) set forth in SEQ ID NO: 1 and encodes an amino acid sequence set forth in SEQ ID NO: 2.

2. A method for controlling male reproductive development of Brassica napus with a homologue of BnOPR3 gene, wherein the BnOPR3.C3 gene has a CDS set forth in SEQ ID NO: 3 and encodes an amino acid sequence set forth in SEQ ID NO: 4.

3. A method for creating a male sterile line of Brassica napus, comprising
inhibiting expression and/or an activity of the BnOPR3.A3 gene according to claim 1 in the Brassica napus, and
selecting a male sterile plant of the Brassica napus.

4. A method for creating a male sterile line of Brassica napus, comprising
inhibiting expression and/or an activity of the BnOPR3.A3 gene according to claim 2 in the Brassica napus, and
selecting a male sterile plant of the Brassica napus.

5. The method according to claim 3, wherein the method is carried out by any manner selected from the group consisting of gene edition, RNA interference, and T-DNA insertion.

6. The method according to claim 4, wherein the method is carried out by any manner selected from the group consisting of gene edition, RNA interference, and T-DNA insertion.

7. The method according to claim 5, wherein the gene edition is carried out by a clustered regularly interspaced short palindromic repeats/CRISPR-associated protein 9 (CRISPR/Cas9) system.

8. The method according to claim 6, wherein the gene edition is carried out by a clustered regularly interspaced short palindromic repeats/CRISPR-associated protein 9 (CRISPR/Cas9) system.

9. The method according to claim 7, wherein the gene edition carried out by a CRISPR/Cas9 system comprises
designing a CRISPR/Cas9 vector target site at an exon 5 on each of the BnOPR3.A3 gene and the BnOPR3.C3 gene, wherein
the vector target site has a DNA sequence set forth in SEQ ID NO: 5.

10. The method according to claim 8, wherein the gene edition carried out by a CRISPR/Cas9 system comprises
designing a CRISPR/Cas9 vector target site at an exon 5 on the BnOPR3.C3 gene, wherein
the vector target site has a DNA sequence set forth in SEQ ID NO: 5.

11. A method for preparing a male sterile line of a BnOPR3 gene, comprising
obtaining the male sterile line of the BnOPR3 gene by the method according to claim 3, and
hybridizing and backcrossing the male sterile line with a target material, such that the target material acquires the BnOPR3 gene mutation and male sterile trait.

12. A method for preparing a male sterile line of a BnOPR3 gene, comprising
obtaining the male sterile line of the BnOPR3 gene by the method according to claim 4, and
hybridizing and backcrossing the male sterile line with a target material, such that the target material acquires the BnOPR3 gene mutation and male sterile trait.

13. A method for hybrid breeding and seed producing of Brassica napus, wherein
hybridizing the male sterile line of the BnOPR3 gene prepared by the method according to claim 11 as a female parent with a target material as a male parent or hybridizing and backcrossing the male sterile line of the BnOPR3 gene prepared by the method according to claim 11 with a target material during the hybrid breeding and the seed producing to allow the target material acquire the male sterile trait of the BnOPR3 gene.

14. A method for hybrid breeding and seed producing of Brassica napus, wherein
hybridizing the male sterile line of the BnOPR3 gene prepared by the method according to claim 12 as a female parent with a target material as a male parent or hybridizing and backcrossing the male sterile line of the BnOPR3 gene prepared by the method according to claim 12 with a target material during the hybrid breeding and the seed producing to allow the target material acquire the male sterile trait of the BnOPR3 gene.
